(19) United States
(12) Patent Application Publication
(10) Pub. No.: US 2018/0353141 A1
(43) Pub. Date: Dec. 13, 2018

(54) SENSOR DISPLAY DEVICE, SENSOR DISPLAY METHOD, AND COMPUTER-READABLE RECORDING MEDIUM

(71) Applicant: FUJITSU LIMITED, Kawasaki-shi (JP)

(72) Inventor: Takayuki Yamaji, Yokahama (JP)

(73) Assignee: FUJITSU LIMITED, Kawasaki-shi (JP)

(21) Appl. No.: 16/106,403

(22) Filed: Aug. 21, 2018

Related U.S. Application Data
(63) Continuation of application No. PCT/JP2016/060040, filed on Mar. 29, 2016.

(51) Int. Cl. A61B 5/00; A61B 5/11

(52) U.S. Cl. CPC A61B 5/743; A61B 5/6804; A61B 5/6898; A61B 2562/0219; A61B 5/4809; A61B 5/4812; A61B 5/1116; A61B 5/1118

(57) ABSTRACT

An information processing device includes: a memory; and a processor coupled to the memory and configured to: specify part of sensor values from among the sensor values which is acquired from a wearable sensor when acquiring the sensor values stored in a storage of the wearable sensor via a read device, the part of sensor values corresponding to a certain time period stretching back from any one of first timing of reading from the wearable sensor, second timing that is due timing at which a user who is associated with the wearable sensor starts duty, and third timing that is due timing at which the user ends duty; and chronologically control a display device to display the part of sensor values or calculated values that are calculated based on the part of sensor values.

### CROSS-REFERENCE TO RELATED APPLICATION

This application is a continuation application of International Application No. PCT/JP2016/060040, filed on Mar. 29, 2016 and designating the U.S., the entire contents of which are incorporated herein by reference.

### FIELD

The embodiments discussed herein are related to a technology to give a display based on a result of measurement performed by a sensor.

### BACKGROUND

A patent document discloses a technology of calculating an activity index and a metabolic index with a device that includes a brain wave sensor and a body motion sensor and displaying the indices as a graph of a time band of 25 hours stretching back from the current time.

Note that, when such a technology is used to estimate the condition of a user, it is not necessarily appropriate to display time series data based on the current time.

Patent Document 1: Japanese Laid-open Patent Publication No. 2006-129887

Patent Document 2: Japanese Laid-open Patent Publication No. 2008-6005

### SUMMARY

According to an aspect of the embodiments, an information processing device includes: a memory; and a processor coupled to the memory and configured to: specify part of sensor values from among the sensor values which is acquired from a wearable sensor when acquiring the sensor values stored in a storage of the wearable sensor via a read device, the part of sensor values corresponding to a certain time period stretching back from any one of first timing of reading from the wearable sensor, second timing that is due timing at which a user who is associated with the wearable sensor starts duty, and third timing that is due timing at which the user ends duty; and chronologically control a display device to display the part of sensor values or calculated values that are calculated based on the part of sensor values.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating an exemplary hardware configuration of a sensor device;

FIG. 2A is a diagram illustrating an exemplary mode of connection;

FIG. 2B is a diagram illustrating an exemplary mode of connection;

FIG. 3 is a diagram representing an exemplary graph;

FIG. 4 is a diagram illustrating an exemplary module configuration of an information processing device;

FIG. 5 is a diagram representing an exemplary first table;

FIG. 6 is a diagram illustrating an exemplary second table;

FIG. 7 is a diagram illustrating an exemplary third table;

FIG. 8 is a diagram illustrating a flow of a main process (A);

FIG. 9 is a diagram illustrating a flow of an acquisition process (A);

FIG. 10 is a diagram illustrating a flow of an acquisition process (B);

FIG. 11 is a diagram illustrating a flow of a first specifying process (A);

FIG. 12 is a diagram illustrating a flow of a first drawing process (A);

FIG. 13 is a diagram representing an exemplary graph in a second embodiment;

FIG. 14 is a diagram illustrating a flow of a main process (B);

FIG. 15 is a diagram illustrating a flow of a second specifying process (A);

FIG. 16 is a diagram illustrating a flow of a first drawing process (B);

FIG. 17 is a diagram representing an exemplary graph in a third embodiment;

FIG. 18 is a diagram illustrating an exemplary first table in the third embodiment;

FIG. 19 is a diagram illustrating a flow of a second specifying process (B);

FIG. 20 is a diagram illustrating a flow of a first drawing process (C);

FIG. 21 is a diagram representing an exemplary graph in a fourth embodiment;

FIG. 22 is a diagram illustrating a flow of a first specifying process (B);

FIG. 23 is a diagram representing an exemplary graph in the fourth embodiment;

FIG. 24 is a diagram representing an exemplary graph in the fourth embodiment;

FIG. 25 is a diagram representing an exemplary graph in a fifth embodiment;

FIG. 26 is a diagram illustrating a flow of a first drawing process (D);

FIG. 27 is a diagram representing an exemplary graph in a sixth embodiment;

FIG. 28 is a diagram illustrating a flow of a first drawing process (E);

FIG. 29 is a diagram representing an exemplary graph in a seventh embodiment;

FIG. 30 is a diagram illustrating a flow of a first specifying process (C);

FIG. 31 is a diagram representing an exemplary graph in the seventh embodiment;

FIG. 32 is a diagram representing an exemplary graph in the seventh embodiment;

FIG. 33 is a diagram representing an exemplary graph in an eighth embodiment;

FIG. 34 is a diagram illustrating a flow of a first drawing process (F);

FIG. 35 is a diagram representing an exemplary graph in a ninth embodiment;

FIG. 36 is a diagram illustrating a flow of a first drawing process (G);

FIG. 37 is a diagram representing an exemplary graph in a tenth embodiment;

FIG. 38 is a diagram illustrating a flow of a first drawing process (H);

FIG. 39 is a diagram representing an exemplary graph in an eleventh embodiment;

FIG. 40 is a diagram illustrating a flow of a first drawing process (I);

FIG. 41 is a diagram representing an exemplary graph in a twelfth embodiment;

FIG. 42 is a diagram illustrating a flow of a main process (C);

FIG. 43 is a diagram representing an exemplary graph in a thirteenth embodiment;

FIG. 44 is a diagram illustrating a flow of a main process (D);

FIG. 45 is a diagram illustrating an exemplary graph in a fourteenth embodiment;

FIG. 46 is a diagram illustrating a flow of a main process (E);

FIG. 47 is a diagram representing an exemplary graph in a fifteenth embodiment;

FIG. 48 is a diagram illustrating a flow of a main process (F); and

FIG. 49 is a functional block diagram of a computer.

### DESCRIPTION OF EMBODIMENT(S)

Preferred embodiments will be explained with reference to accompanying drawings. However, the invention is not limited by these embodiments.

### [a] First Embodiment

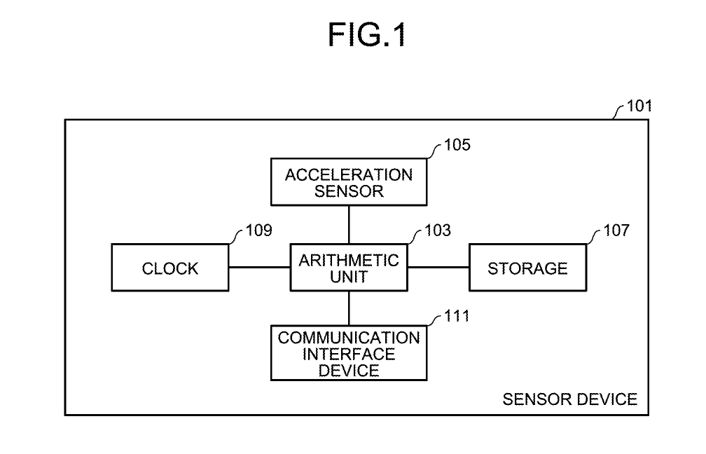

FIG. 1 illustrates an exemplary hardware configuration of a sensor device 101. In a state where a user wears the sensor device 101, the sensor device 101 measures acceleration. For example, the sensor device 101 is attached to a belt or pants and is worn in the position of the waist of the user. Note that the sensor device 101 may be worn in another part (such as the head, neck, chest, belly, back, an arm or a leg). Instead of the sensor device 101, a wearable terminal or a mobile phone terminal may be used.

The sensor device 101 includes an arithmetic unit 103, an acceleration sensor 105, a storage 107, a clock 109 and a communication interface device 111. The arithmetic unit 103 performs various types of arithmetic processing. The acceleration sensor 105 measures acceleration. The storage 107 stores various types of data and programs. The clock 109 counts dates. The communication interface device 111 is, for example, a radio integrated circuit (IC) tag or a universal serial bus (USB) interface device. The communication interface device 111 may be another interface device according to near field communication.

The sensor device 101 accumulates acceleration data to which the dates of measurement are added, that is, acceleration time series data. The sensor device 101 outputs the acceleration time series data via the communication interface device 111.

The sensor device 101 may calculate amounts of activity of the user based on the acceleration time series data. The sensor device 101 may output time series data about the calculated amounts of activity via the communication interface device 111. A method of calculating amounts of activity accords with related technologies.

The sensor device 101 may determine body positions of the user based on the acceleration time series data. The sensor device 101 may output time series data about the determined body positions via the communication interface device 111. A method of determining body positions accords with related technologies. The sensor device 101 may include a sensor other than the acceleration sensor 105 as long as the sensor is capable of measuring activities of the user and a state of sleep. Exemplary sensors include a radio-frequency sensor capable of detecting motions of the body, such as the heart or lungs, a sensor capable of detecting pulsation or breathing, a radio-frequency sensor capable of detecting motion of the body itself and an image sensor. In that case, the sensor device 101 may accumulate time series data about heart rates, breathing rates and/or time series data about body motions and output the time series data via the communication interface device 111. Alternatively, the measured data may be output via the communication interface device 111 and another computer may detect heart rates, breathing rates and/or body motions and they may be accumulated as time series data.

The user who wears the sensor device 101 may be, for example, a driver of a train or an automobile. In this example, a manager understands the condition of activity of a driver whose is going to engage in operations to implement safe operations.

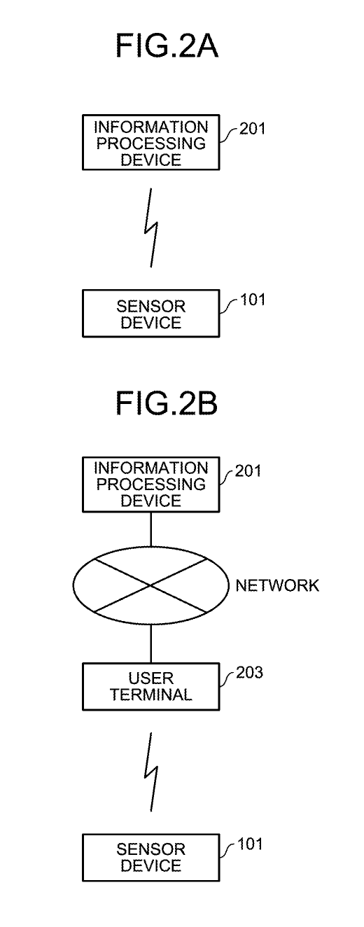

Thus an information processing device that is used by the manager reads the data accumulated in the sensor device 101 and displays the data. FIG. 2A illustrates an exemplary mode of connection. In this example, an information processing device 201 reads data directly from the sensor device 101. The sensor device 101 stores the time at which data is read.

Another mode of connection like that illustrated in FIG. 2B may be possible. A user terminal 203 is caused to temporarily read the data accumulated in the sensor device 101 and the data is transferred from the user terminal 203 to the information processing device 201. Thus the user terminal 203 includes a read device corresponding to the communication interface device 111 of the sensor device 101 and transfers data via a network. The network is, for example, the Internet, a dedicated line or a local area network (LAN). The user terminal 203 is, for example, a mobile phone terminal or a tablet terminal. The user terminal 203 stores the time of reading and transmits the time together with the data to transfer. FIG. 2B illustrates an example where the user terminal 203 and the information processing device 201 are devices different from each other; however, embodiments need not be limited to such a mode of connection. For example, the function of the information processing device 201 to execute the embodiment may be implemented in the user terminal 203.

On receiving an instruction to display a graph from the manager, the information processing device 201 displays a graph representing the amounts of activity in 24 hours stretching back from the time when the data is read.

For example, in a case of an outward journey of a driver on duty from a service office to a destination, reading data in the connection mode illustrated in FIG. 2A at a time of departure enables the manger to check later the condition of the driver at the time of departure. Reading data in the connection mode illustrated in FIG. 2B at a time of arrival at the destination enables the manager to check later the condition of the driver during driving.

On the other hand, in a case of a homeward journey from the destination to the service office, reading data in the connection mode illustrated in FIG. 2B at the time of departure enables the manger to check later the condition of the driver at the time of departure. Reading data in the connection mode illustrated in FIG. 2A at the time of arrival at the service office enables the manager to check later the condition of the driver during driving.

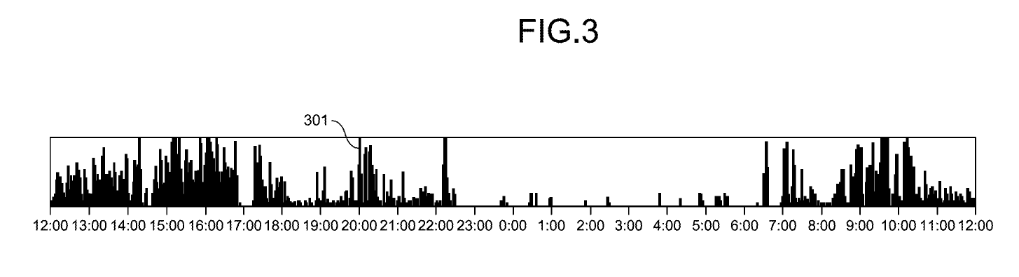

FIG. 3 represents an exemplary graph. The end on the right corresponds to a time point of reading. A bar graph 301 represents the amounts of activity from a time point that is 24 hours back from the time of reading. For example, when the driver departs at 12:00, the manager is able to know how the driver acted before the departure. On the other hand, when the driver arrives at 12:00, the manager is able to know at which timing the driver took a rest midway.

A user other than drivers, such as a machine operator, a surveillant or a medical representative, may use the sensor device 101. Descriptions of the outline of the first embodiment end here.

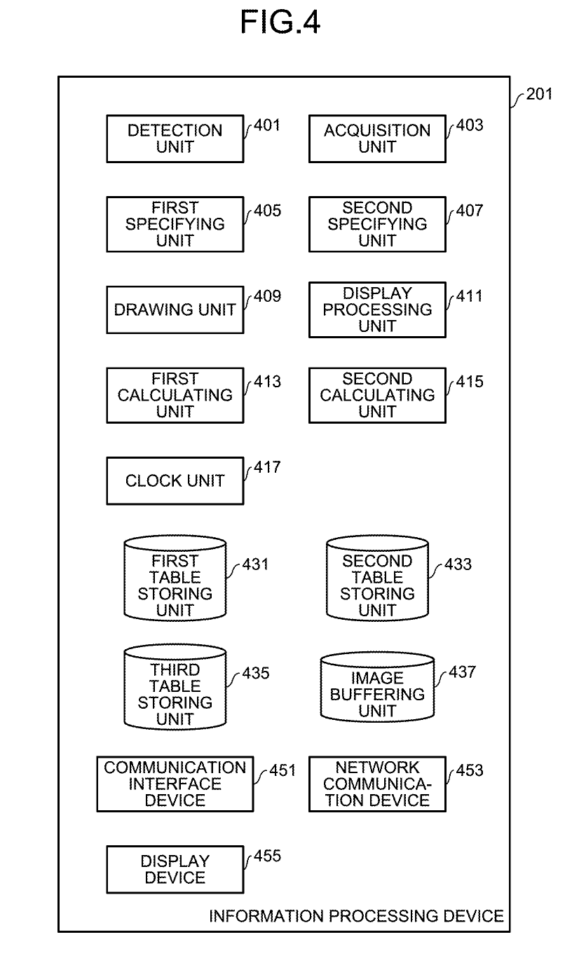

Operations of the information processing device 201 will be described. FIG. 4 illustrates an exemplary module configuration of the information processing device 201. The information processing device 201 includes a detection unit 401, an acquisition unit 403, a first specifying unit 405, a second specifying unit 407, a drawing unit 409, a display processing unit 411, a first calculating unit 413, a second calculating unit 415, a clock unit 417, a first table storing unit 431, a second table storing unit 433, a third table storing unit 435, an image buffering unit 437, a communication interface device 451, a network communication device 453 and a display device 455.

The detection unit 401 detects the sensor device 101. The acquisition unit 403 acquires time series data. The first specifying unit 405 specifies a display period. The second specifying unit 407 specifies a period of sleep. The drawing unit 409 draws a graph. The display processing unit 411 performs a process of displaying display parts, such as a graph and a box. The display process performed by the display processing unit 411, for example, implements display of an image of a graph that is stored in the image buffering unit 437 on the display device 455. The display processing unit 411 is an exemplary output processing unit. The first calculating unit 413 calculates duration of a first period from a time point when the user awakes until a due time of start of duty. The second calculating unit 415 calculates duration of a second period from the time point when the user awakes until a due time of end of duty. The clock unit 417 measures the current time.

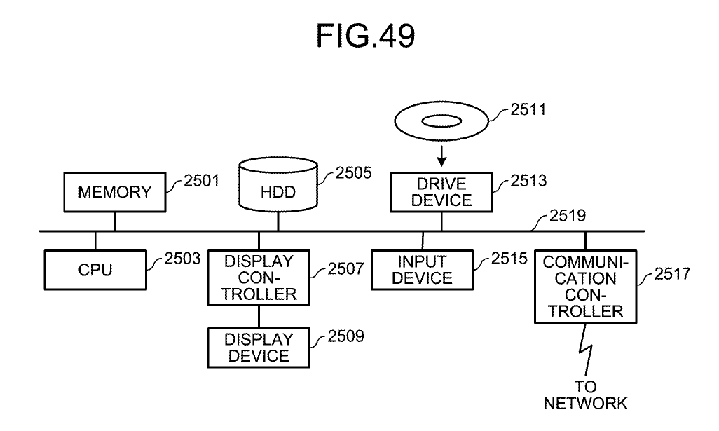

The detection unit 401, the acquisition unit 403, the first specifying unit 405, the second specifying unit 407, the drawing unit 409, the display processing unit 411, the first calculating unit 413, the second calculating unit 415 and the clock unit 417 described above are realized using hardware resources (such as those in FIG. 49) and a program that causes a processor to execute processes to be described below.

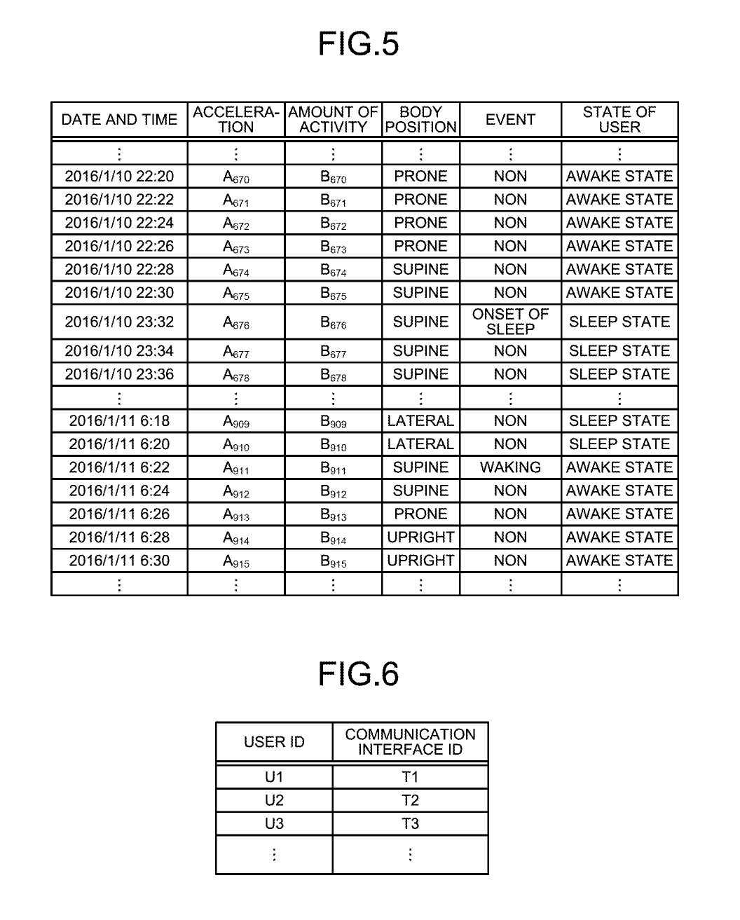

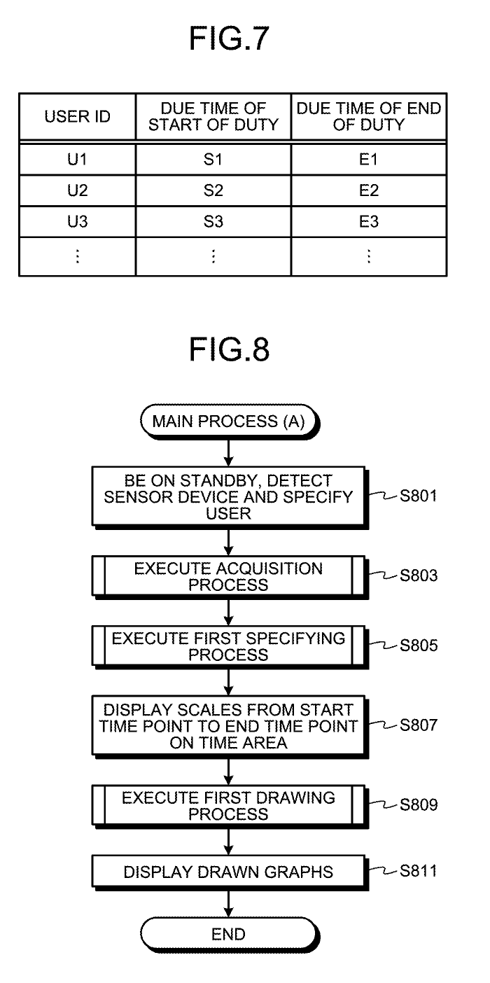

The first table storing unit 431 stores a first table. The first table will be described below using FIG. 5. The second table storing unit 433 stores a second table. The second table will be described below using FIG. 6. The third table storing unit 435 stores a third table. The third table will be described below using FIG. 7. The image buffering unit 437 stores the image of the drawn graph.

The first table storing unit 431, the second table storing unit 433, the third table storing unit 435 and the image buffering unit 437 described above are realized using hardware resources (such as those in FIG. 49).

The communication interface device 451 implements communication with the communication interface device 111 of the sensor device 101. The communication interface device 451 is, for example, a wireless IC tag read device or a USB interface. The communication interface device 451 may be an interface device according to other near field communication. The network communication device 453 implements communication via the network. The display device 455 performs the display process to display, for example, a graph or a box. The display device 455 has a general function of displaying an image and may be a general-purpose device.

FIG. 5 represents an exemplary first table. The first table of the example has records corresponding to the time points of measurement of acceleration. The records of the first table include a field in which dates are stored, a field in which acceleration is stored, a field in which amounts of activity are stored, a field in which body positions are stored, a field in which events are stored and a field in which states of the user are stored.

A date specifies timing when acceleration is measured. A body position is, for example, a prone position, a supine position, a lateral position or an upright position. An event is onset of sleep or waking. A state of the user is an awake state or a sleep state. Note that the onset of sleep means switching from the awake state to the sleep state. Waking means switching from the sleep state to the awake state.

FIG. 6 represents an exemplary second table. The second table has records corresponding to users (such as drivers). The records of the second table include a field in which user IDs are stored and a filed in which communication interface IDs are stored. A communication interface ID identifies the communication interface device 111 of the sensor device 101 that is used by the user. The second table is provided in advance before the process of the first embodiment is executed.

FIG. 7 represents an exemplary third table. The third table has records corresponding to the users. The records of the third table have a field in which the user IDs are stored, a field in which due times of start of duty are stored and a field in which due times of end of duty are stored. The third table is provided in advance before processes to be described below are executed. Note that the third table is used in a fourth embodiment and embodiments following the fourth embodiment.

Processes performed by the information processing device 201 will be described. FIG. 8 illustrates a flow of a main process (A). First of all, the case of the connection mode illustrated in FIG. 2A will be described. The detection unit 401 in on standby, then detects the sensor device 101 and specifies the user (S801). Specifically, the communication interface device 451 of the information processing device 201 implements communication with the communication interface device 111 of the sensor device 101. Based on the second table, the detection unit 401 specifies a user ID corresponding to the ID of the communication interface device 111 that is received in the communication.

When the sensor device 101 is detected, the acquisition unit 403 executes an acquisition process (S803). In the acquisition process, time series data that is used for analysis is obtained from the sensor device 101.

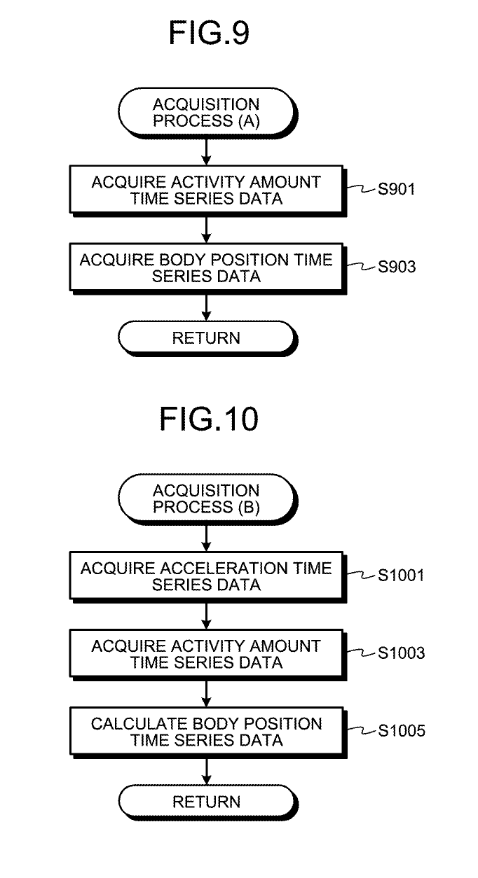

FIG. 9 illustrates a flow of an acquisition process (A). The acquisition process (A) presupposes that the sensor device 101 has calculated amounts of activity and further has determined body positions. The acquisition unit 403 acquires time series data about the amounts of activity via the communication interface device 451 (S901). Furthermore, the acquisition unit 403 acquires time series data about the body positions via the communication interface device 451 (S903). When a display is given based on only the activity amount time series data, the body position time series data need not be acquired. On ending the acquisition process (A), return to the main process (A) that is the caller process.

Instead of the acquisition process (A) represented in FIG. 9, an acquisition process (B) may be executed. In the acquisition process (B), only the acceleration time series data is acquired. Accordingly, the sensor device 101 need not calculate amounts of activity. Furthermore, the sensor device 101 need not determine body positions.

FIG. 10 illustrates a flow of the acquisition process (B). The acquisition unit 403 acquires the acceleration time series data via the communication interface device 451 (S1001). The second specifying unit 407 calculates activity amount time series data based on the acceleration time series data (S1003). A method of calculating amounts of activity accords with related technologies. Furthermore, the second specifying unit 407 calculates body position time series data based on the acceleration time series data (S1005). A method of determining body positions accords with related technologies. When analysis is perfoLmed based on only the activity amount time series data, the body position time series data need not necessarily be calculated. On ending the acquisition process (B), return to the main process (A) that is the caller process. The data that is acquired by performing the acquisition process (A) or the acquisitions process (B) is stored in the first table storing unit 431 that is exemplified in FIG. 5.

Return to descriptions of FIG. 8. In the case of the connection mode illustrated in FIG. 2A, at this time point, move to a standby state temporarily, and then wait for an instruction from the manager and move to the processes at S805 and the following steps.

The case of connection mode illustrated in FIG. 2B will be described. In this case, the process at S801 is omitted. At the time point when an instruction from the manager is received, the processes from S803 are started.

In this case, in the acquisition process at S803, the time series data and the time of reading are received via the network communication device 453. In the case of the acquisition process (A), the mode of acquisition at S901 and S903 is reception of data. In the case of the acquisition process (B), the mode of acquisition at S1001 is reception of data. Here, for example, the user is specified by receiving the user ID.

Then directly move to the process at S805 illustrated in FIG. 8. The processes at S805 and the following steps are the same in any of the connection modes.

The first specifying unit 405 executes a first specifying process (S805). In the first specifying process, a display period is specified.

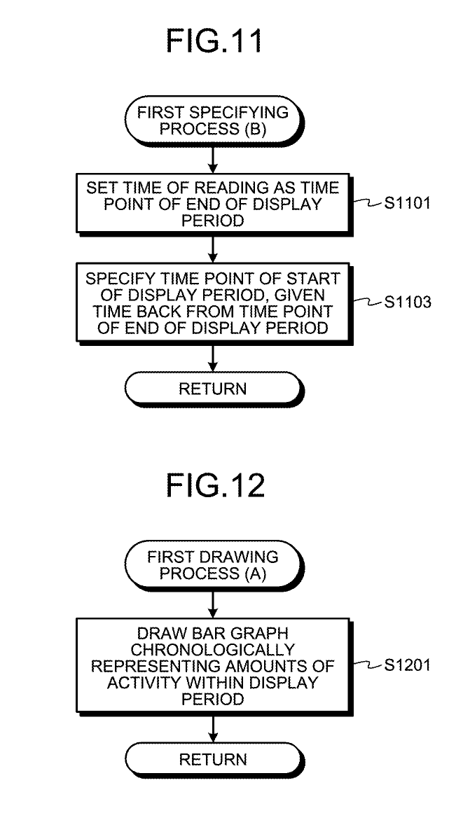

FIG. 11 illustrates a flow of the first specifying process (A). The first specifying unit 405 sets the time of reading as a time point of the end of the display period (S1101). The first specifying unit 405 specifies a time point of the start of the display period, a given time (24 hours in this example) back from the time point of the end of the display period (S1103). On ending the first specifying process, return to the main process (A) that is the caller process.

Return to descriptions of FIG. 8. The display processing unit 411 displays time scales from the start time point to the end time point in a time area on a display screen (S807). Specifically, the display processing unit 411 causes the display device 455 to display the time scales.

The drawing unit 409 executes a first drawing process (S809). In the first drawing process, the drawing unit 409 performs drawing in a first graph area.

FIG. 12 illustrates a flow of the first drawing process (A). The drawing unit 409 draws the bar graph 301 chronologically representing the amounts of activity within the display period (S1201). The drawn graph is accumulated in the image buffering unit 437. On ending the first drawing process (A), return to the main process (A) that is the caller process.

Return to descriptions of FIG. 8. The display processing unit 411 displays the drawn graph (S811). In the first embodiment, the display processing unit 411 causes the display device 455 to display the bar graph 301 that is drawn in the first drawing process (A). Then the main process ends. In the following embodiments, each of graphs drawn in the first drawing process and the second drawing process is displayed at S811.

According to the first embodiment, regardless of the timing to display a graph, it is possible to display amounts of activity in the past based on the time when data is read.

### [b] Second Embodiment

In a second embodiment, a period of sleep is displayed by a band graph.

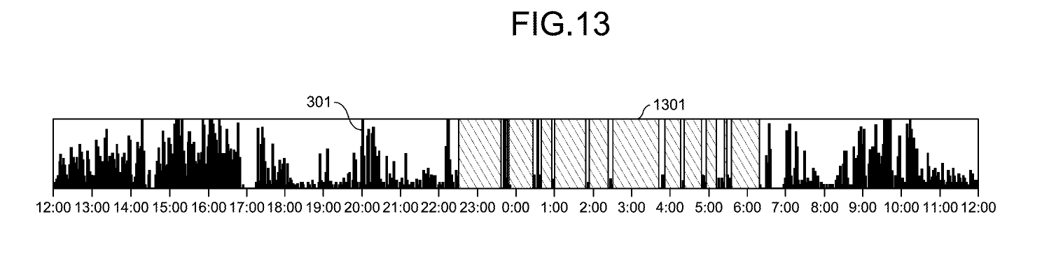

FIG. 13 represents an exemplary graph in the second embodiment. A band graph 1301 represents a period of sleep.

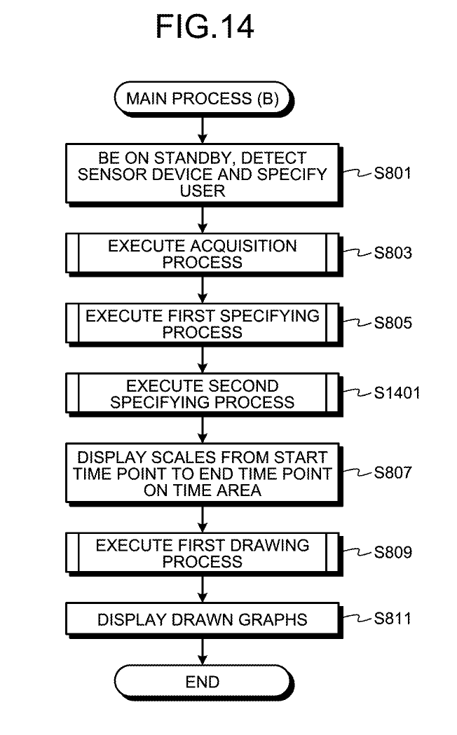

In the second embodiment, instead of the main process (A), a main process (B) is executed. FIG. 14 illustrates the main process (B). The processes from S801 to S805 are the same as those in FIG. 8. The second specifying unit 407 executes the second specifying process (S1401). In the second specifying process, periods of sleep are specified.

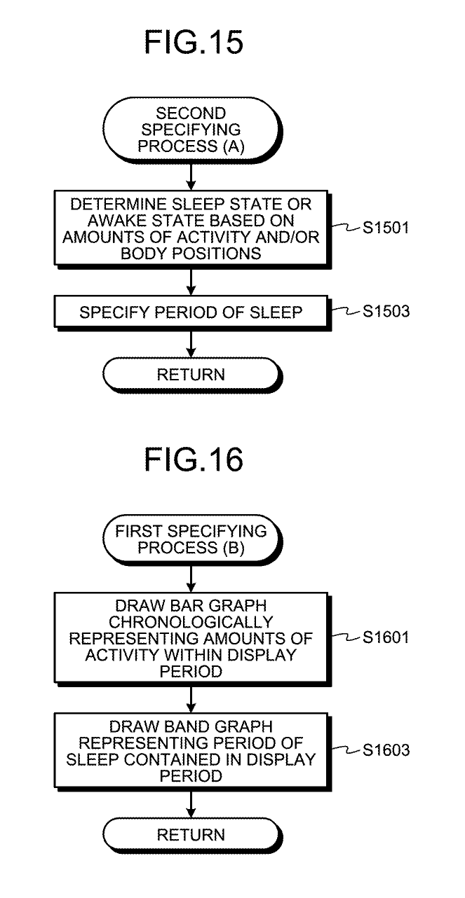

FIG. 15 illustrates a flow of the second specifying process (A). The second specifying unit 407 determines, with respect to each time point of measurement, in which of the sleep state and the awake state the user is based on the activity amount time series data and/or the body position time series data (S1501). When a sensor other than acceleration sensors is used as described above, the second specifying unit 407 may determine in which of the sleep state and the awake state the user is from time series data about heart rates, breathing rates and/or body motions. The second specifying unit 407 specifies periods of sleep during each of which the sleep state continues (S1503). On ending the second specifying process (A), return to the main process (B) that is the caller process.

Return to descriptions of FIG. 14. In the second embodiment, at step S809, a first drawing process (B) is executed instead of the first drawing process (A).

FIG. 16 illustrates a flow of the first drawing process (B). The drawing unit 409 draws the bar graph 301 chronologically representing amounts of activity within the display period (S1601). The drawing unit 409 draws the band graph 1301 representing periods of sleep contained in the display period (S1603). The drawn graphs are both stored in the image buffering unit 437. On ending the first drawing process (B), return to the main process (B) that is the caller process.

Return to descriptions of FIG. 14. At S811, the display processing unit 411 displays the drawn graphs. In the second embodiment, the display device 455 is caused to display the bar graph 301 and the band graph 1301 that are drawn in the first drawing process (B).

According to the second embodiment, regardless of the timing of display of the graphs, it is possible to display the periods of sleep in the past based on the time when data is read.

### [c] Third Embodiment

In a third embodiment, modes of display are discriminated according to depths of sleep.

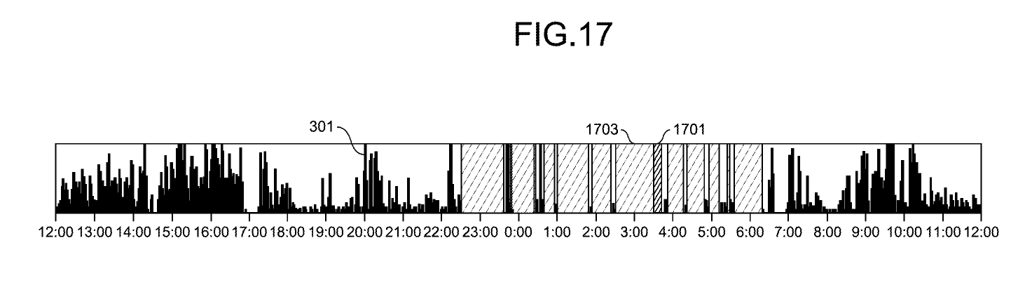

FIG. 17 represents an exemplary graph in the third embodiment. A band graph 1701 represents a band of time of light sleep (referred to as first time band below). A band graph 1703 represents bands of time of deep sleep (referred to as second time bands below).

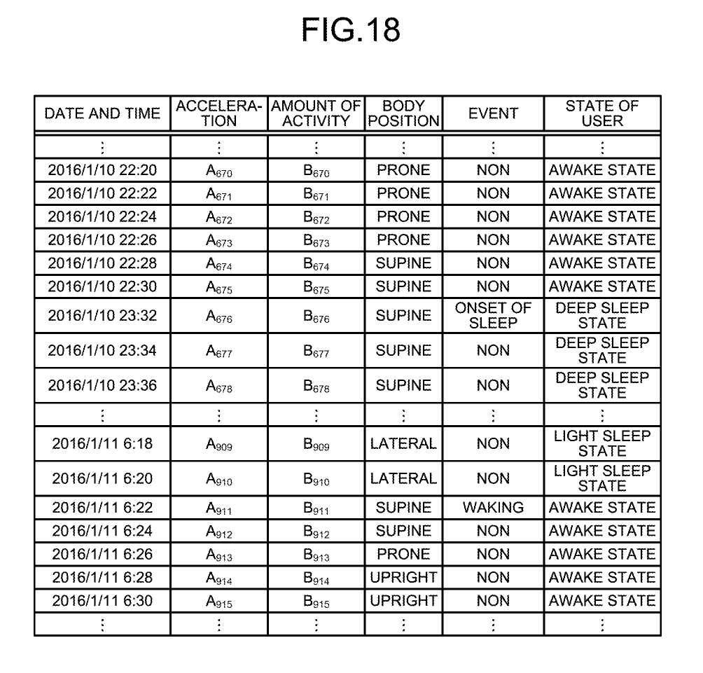

FIG. 18 illustrates an exemplary first table in the third embodiment. In the third embodiment, the sleep state is divided into a light sleep state and a deep sleep state.

In the third embodiment, the main process (B) is executed. At S805, the first specifying process (A) is executed. At S1401, a second specifying process (B) is executed instead of the second specifying process (A).

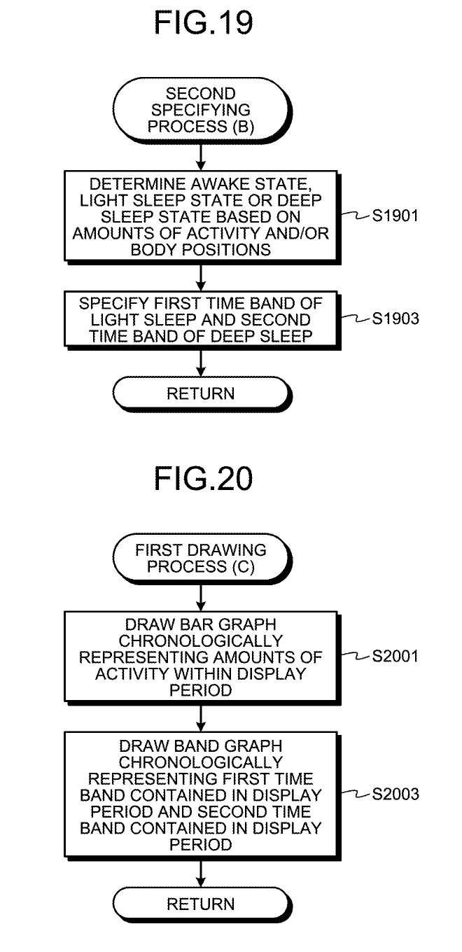

FIG. 19 illustrates a flow of the second specifying process (B). The second specifying unit 407 determines, with respect to each time point of measurement, in which any one of the awake state, the light sleep state and the deep sleep state the user is based on the activity amount time series data and/or the body position time series data (S1901). The second specifying unit 407 specifies the first time band in which the light sleep state continues and the second time bands in which deep sleep continues (S1903). On ending the second specifying process (B), return to the main process (B) that is the caller process.

Return to descriptions of FIG. 14. In the third embodiment, at step S809, a first drawing process (C) is executed.

FIG. 20 illustrates a flow of the first drawing process (C). The drawing unit 409 draws the bar graph 301 chronologically representing the amounts of activity within the display period (S2001). The drawing unit 409 draws the band graph 1701 representing the first time band contained in the display period and the band graph 1703 representing the second time bands (S2003). The drawn graphs are both stored in the image buffering unit 437. On ending the first drawing process (C), return to the main process (B) that is the caller process.

Return to descriptions of FIG. 14. At step S811, the display processing unit 411 displays the drawn graphs. In the third embodiment, the display processing unit 411 causes the display device 455 to display the bar graph 301, the band graph 1701 and the band graph 1703 that are drawn in the first drawing process (C).

According to the third embodiment, regardless of the timing of display of the graphs, it is possible to represent the sleep states in the past based on the time when data is read.

### [d] Fourth Embodiment

In a fourth embodiment, a graph is displayed based on a due time of the start of duty. In the following embodiments, the user causes the data of the sensor device 101 to be read before the start of duty.

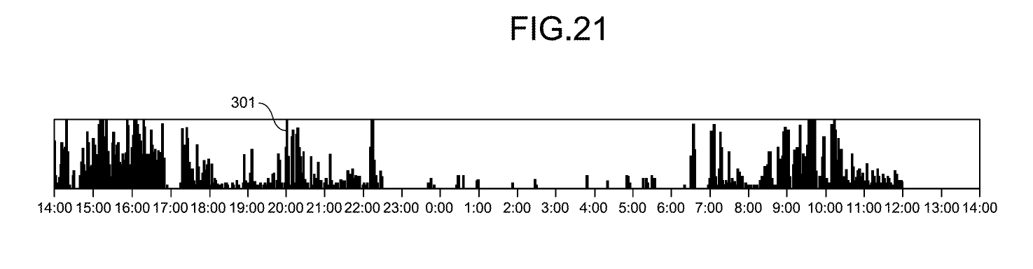

FIG. 21 represents an exemplary graph in the fourth embodiment. The end on the right corresponds to a due time of the start of duty (14:00 in the example). The bar graph 301 represents the amounts of activities from a time point, 24 hours back from the due time of the start of duty. For example, the manager easily imagines the condition before departure of the driver.

In the fourth embodiment, the main process (A) is executed. At S805 illustrated in FIG. 8, a first specifying process (B) is executed.

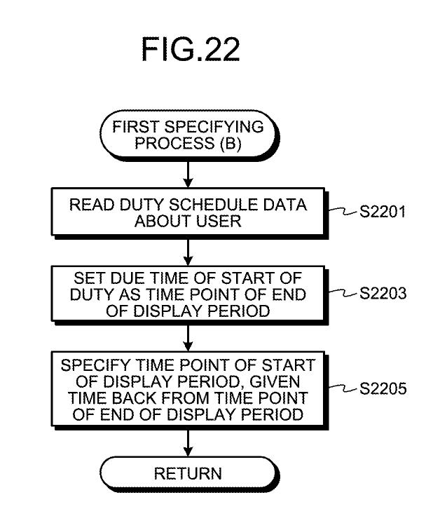

FIG. 22 illustrates a flow of the first specifying process (B). The first specifying unit 405 reads duty schedule data about the user from the first table (S2201). The first specifying unit 405 sets the due time of the start of duty as the time point of the end of the display period (S2203). The first specifying unit 405 specifies a time point of the start of the display period, a given time back from the time point of the end of the display period (S2205). On ending the first specifying process (B), return to the main process (A) that is the caller process.

At S809 illustrated in FIG. 8, the first drawing process (A) is executed. At S811, the display processing unit 411 displays the drawn graph. In the fourth embodiment, the display processing unit 411 causes the display device 455 to display the bar graph 301 that is drawn in the first drawing process (A).

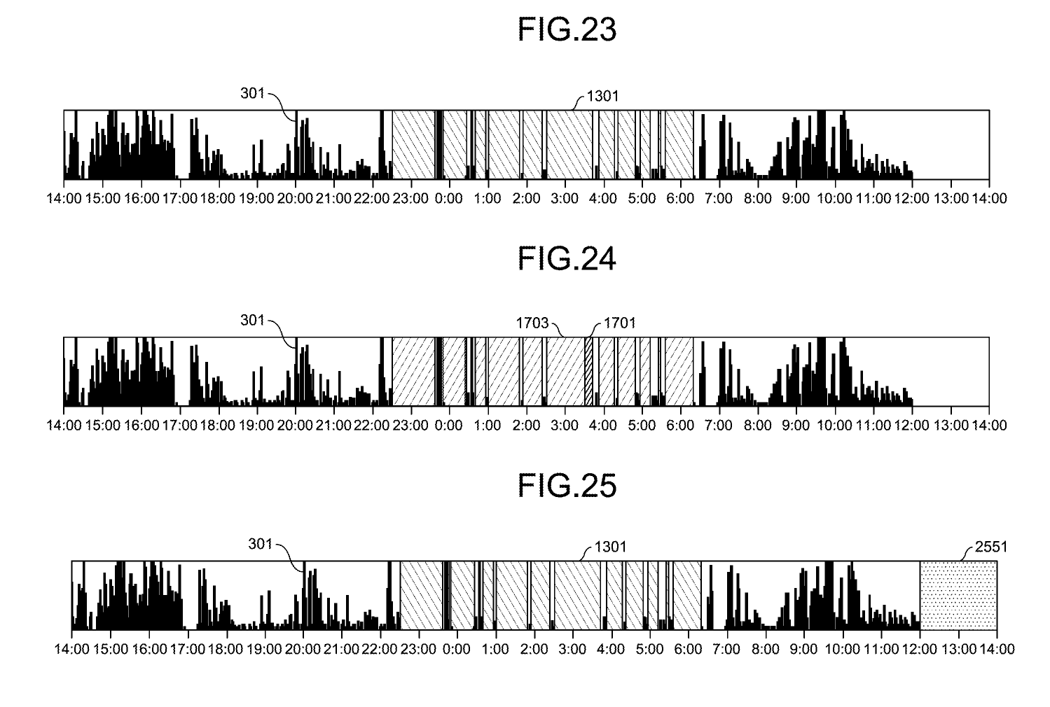

Another exemplary graph will be represented. FIG. 23 represents an exemplary graph obtained in a case where, in the main process (B), the first specifying process (B) is executed at S805, the second specifying process (A) is executed at S1401 and the first drawing process (B) is executed at S809.

FIG. 24 represents an exemplary graph obtained in a case where, in the main process (B), the first specifying process (B) is executed at S805, the second specifying process (B) is executed at S1401 and the first drawing process (C) is executed at S809.

According to the fourth embodiment, the period stretching back from the start of duty is easily known. For example, the period from the time point when the user awakes until the start of duty is easily known.

### [e] Fifth Embodiment

In a fifth embodiment, a standby period until the start of duty is represented by a band graph.

FIG. 25 represents an exemplary graph in the fifth embodiment. A band graph 2551 represents a standby period until the start of duty (12:00 to 14:00 in this example). Visualizing the standby period, for example, enables the manager to easily imagine how the body condition of the driver will change based on the due time of departure.

In the firth embodiment, the main process (B) is executed. The first specifying process (B) is executed at S805 and the second specifying process (A) is executed at S1401. At S809, a first drawing process (D) is executed.

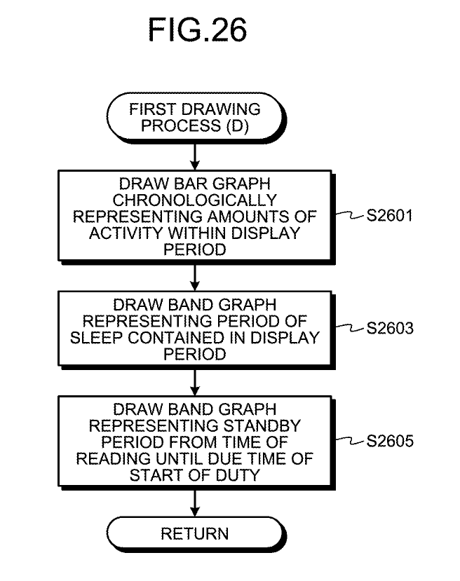

FIG. 26 illustrates a flow of the first drawing process (D). The drawing unit 409 draws the bar graph 301 chronologically representing amounts of activity within the display period (S2601). The drawing unit 409 draws the band graph 1301 representing the periods of sleep contained in the display period (S2603). The drawing unit 409 draws the band graph 2551 representing the standby period from the time of reading until the due time of the start of duty (S2605). On ending the first drawing process (D), return to the main process (B) that is the caller process.

At S811, the display processing unit 411 displays the drawn graphs. In the fifth embodiment, the display device 455 is caused to display the bar graph 301, the band graph 1301 and the band graph 2551 that are drawn in the first drawing process (D).

The example where the standby period is represented by the bar graph has been described. Alternatively, the standby period may be displayed in another mode. For example, the start and end of the standby period may be marked. Alternatively, a line discriminating the standby period may be drawn.

The fifth embodiment is utilized to predict the effect of the standby on the body condition based on the period during which the user is on standby.

### [f] Sixth Embodiment

In a sixth embodiment, the first time band and the second time bands are discriminated and the standby period until the start of duty is represented by the band graph 2551.

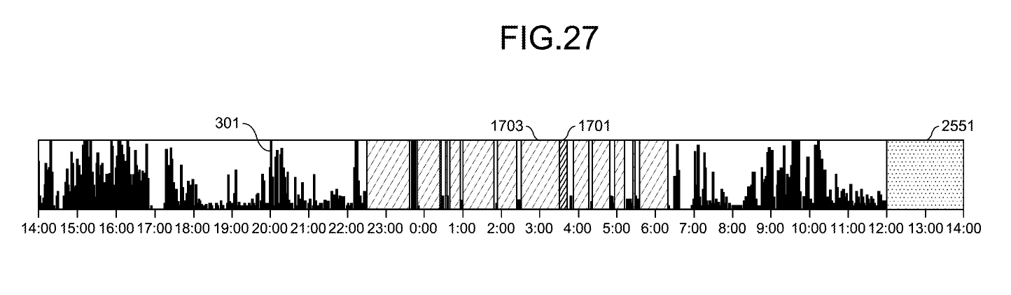

FIG. 27 represents an exemplary graph in the sixth embodiment. In addition to the bar graph 301, the band graph 1701, the band graph 1703 and the band graph 2551 are represented.

In the sixth embodiment, the main process (B) is executed. Furthermore, the first specifying process (B) is executed at S805 and the second specifying process (B) is executed at S1401. At S809, a first drawing process (E) is executed.

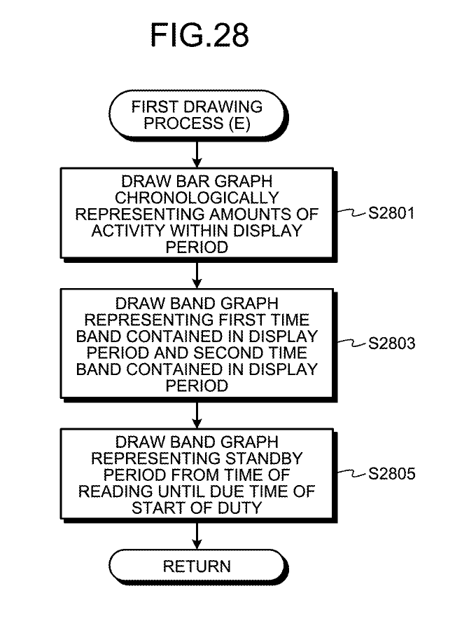

FIG. 28 represents a flow of the first drawing process (E). The drawing unit 409 draws the bar graph 301 chronologically representing amounts of activity within the display period (S2801). The drawing unit 409 draws the band graph 1701 representing the first time band and the band graph 1703 representing the second time bands (S2803). The drawing unit 409 draws the band graph 2551 representing the standby period from the time of reading until the due time of the start of duty (S2805). On ending the first drawing process (E), return to the main process (B) that is the caller process.

At S811, the display processing unit 411 displays the drawn graphs. In the sixth embodiment, the display processing unit 411 causes the display device 455 to display the bar graph 301, the band graph 1701, the band graph 1703 and the band graph 2551 that are drawn in the first drawing process (E).

According to the sixth embodiment, the depth of sleep can be taken into consideration to predict the effect on the body condition based on the period during which the user is on standby.

### [g] Seventh Embodiment

In a seventh embodiment, a graph is displayed based on a due time of the end of duty.

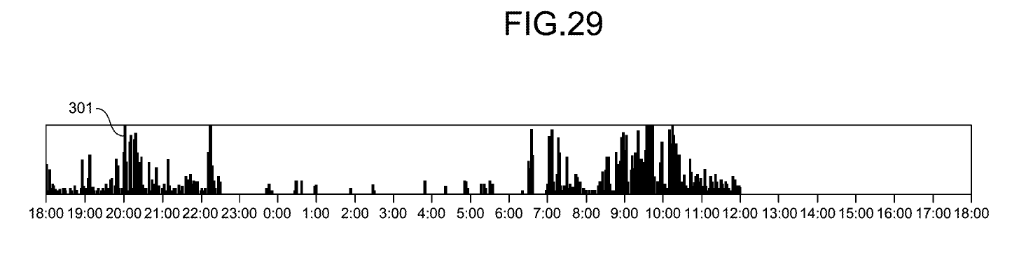

FIG. 29 represents an exemplary graph in the seventh embodiment. The end on the right corresponds to a due time of the end of duty (18:00 in the example). The bar graph 301 represents the amounts of activity from a time point, 24 hours back from the due time of the end of duty. For example, the manager easily imagines the body condition before arrival of the driver at a destination.

In the seventh embodiment, the main process (A) is executed. At S805, a first specifying process (C) is executed.

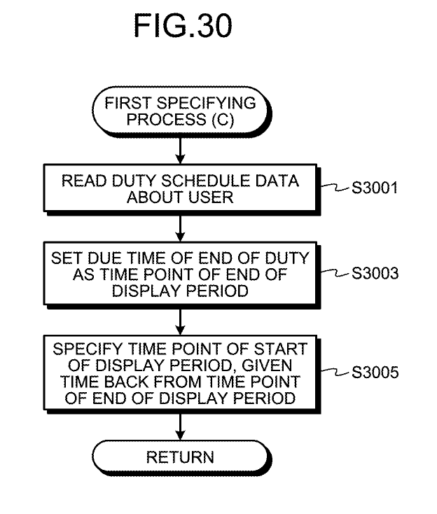

FIG. 30 illustrates a flow of the first specifying process (C). The first specifying unit 405 reads duty schedule data about the user (S3001). The first specifying unit 405 sets the due time of the end of duty as a time point of the end of the display period (S3003). The first specifying unit 405 specifies a time point of the start of the display period, a given time back from the time point of the end of the display period (S3005). On ending the first specifying process (C), return to the main process (A) that is the caller process.

At S809, the first drawing process (A) is executed.

At S811, the display processing unit 411 displays the drawn graph. In the fourth embodiment, the display processing unit 411 causes the display device 455 to display the bar graph 301 that is drawn in the first drawing process (A).

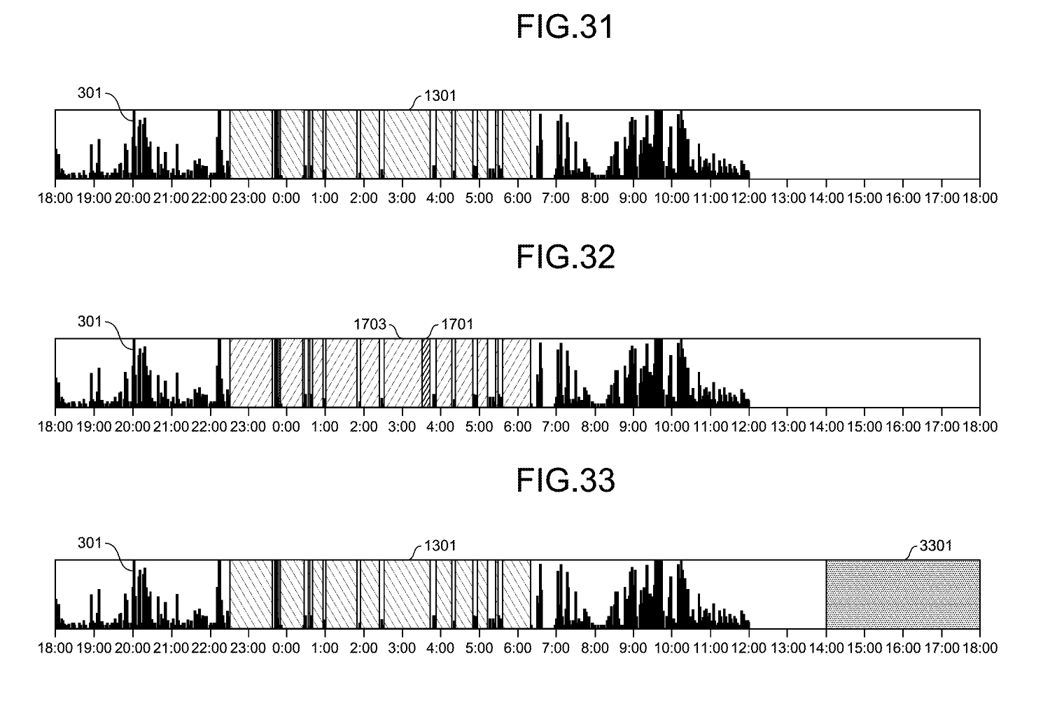

Another exemplary graph will be represented. FIG. 31 represents an exemplary graph obtained in a case where, in the main process (B), the first specifying process (C) is executed at S805, the second specifying process (A) is executed at S1401 and the first drawing process (B) is executed at S809.

FIG. 32 represents an exemplary graph obtained in a case where, in the main process (B), the first specifying process (C) is executed at S805, the second specifying process (B) is executed at S1401 and the first drawing process (C) is executed at S809.

According to the seventh embodiment, the period stretching back from the end of duty is easily known. For example, the period from the time point when the user awakes until the end of duty is easily known.

### [h] Eighth Embodiment

In an eighth embodiment, a period of duty is represented by a band graph.

FIG. 33 represents an exemplary graph in the eighth embodiment. A band graph 3301 represents a period of duty (14:00 to 18:00 in this example). Visualizing the period of duty enables the manager to easily imagine how the body condition of the driver will change during driving.

In the eighth embodiment, the main process (B) is executed. The first specifying process (C) is executed at S805 and the second specifying process (A) is executed at S1401. At S809, a first drawing process (F) is executed.

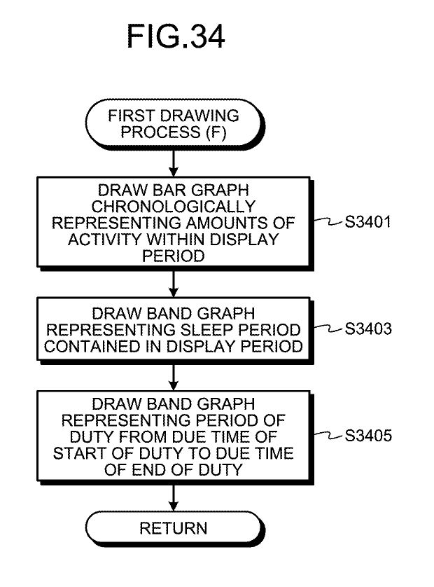

FIG. 34 illustrates a flow of the first drawing process (F). The drawing unit 409 draws the bar graph 301 chronologically representing amounts of activity within the display period (S3401). The drawing unit 409 draws the band graph 1301 representing periods of sleep contained in the display period (S3403). The drawing unit 409 draws the band graph 3301 representing the period of duty from a due time of the start of duty until a due time of the end of duty (S3405). On ending the first drawing process (F), return to the main process (A) that is the caller process.

At S811, the display processing unit 411 displays the drawn graphs. In the eighth embodiment, the display processing unit 411 causes the display device 455 to display the bar graph 301, the band graph 1301 and the band graph 3301 that are drawn in the first drawing process (F).

The example where the period of duty is represented by the bar graph has been described. Alternatively, the period of duty may be displayed in another mode. For example, the start and end of the period of duty may be marked. Alternatively, a line discriminating the period of duty may be drawn.

The eighth embodiment is utilized to predict the body condition of the user on duty based on the period during which the user in on duty.

### [i] Ninth Embodiment

In a ninth embodiment, the first time band and the second time bands are discriminated and a period of duty is represented by the band graph 3301.

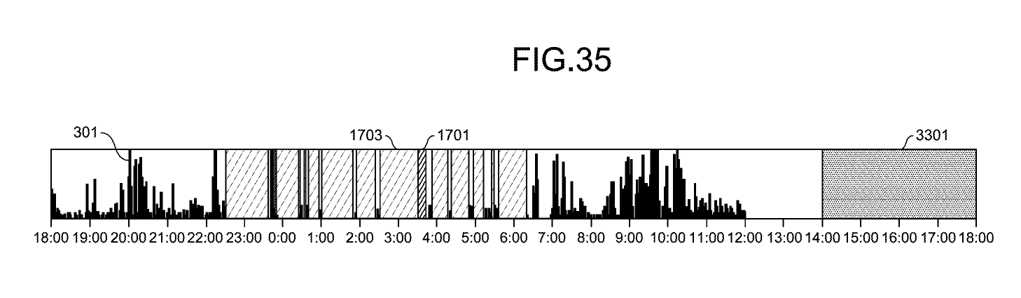

FIG. 35 represents an exemplary graph in the ninth embodiment. In addition to the bar graph 301, the band graph 1701, the band graph 1703 and the band graph 3301 are represented.

In the ninth embodiment, the main process (B) is executed. Furthermore, the first specifying process (C) is executed at S805 and the second specifying process (B) is executed at S1401. At S809, a first drawing process (G) is executed.

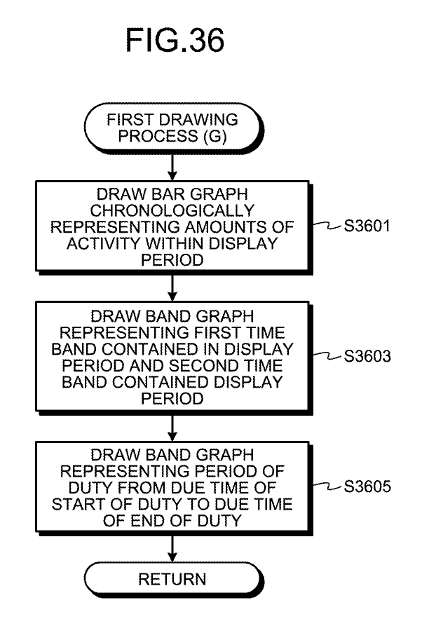

FIG. 36 represents a flow of the first drawing process (G). The drawing unit 409 draws the bar graph 301 chronologically representing amounts of activity within the display period (S3601). The drawing unit 409 draws the band graph 1701 representing the first time band and contained in the display period and the band graph 1703 representing the second time bands (S3603). The drawing unit 409 draws the band graph 3301 representing the period of duty from a due time of the start of duty until a due time of the end of duty (S3605). On ending the first drawing process (G), return to the main process (B) that is the caller process.

At S811, the display processing unit 411 displays the drawn graphs. In the ninth embodiment, the display processing unit 411 causes the display device 455 to display the bar graph 301, the band graph 1701, the band graph 1703 and the band graph 3301 that are drawn in the first drawing process (G).

According to the ninth embodiment, the depth of sleep can be taken into consideration to predict the body condition of the user on duty.

### [j] Tenth Embodiment

In a tenth embodiment, a standby period until the start of duty is represented by the band graph 2551 and furthermore a period of duty is represented by the band graph 3301.

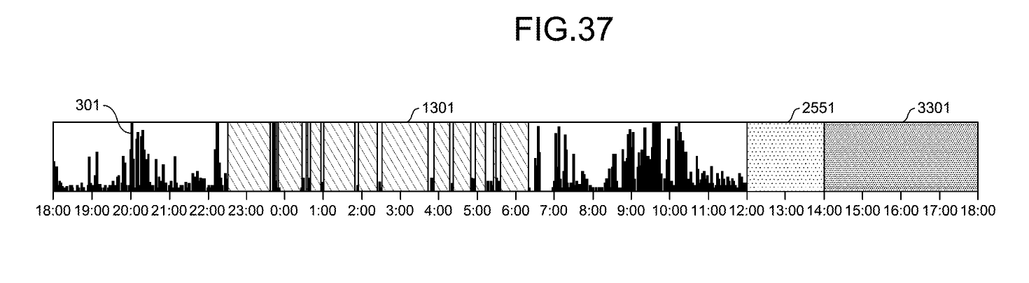

FIG. 37 represents an exemplary graph in the tenth embodiment. In addition to the bar graph 301, the band graph 1301, the band graph 2551 and the band graph 3301 are represented.

In the tenth embodiment, the main process (B) is executed. The first specifying process (C) is executed at S805 and the second specifying process (A) is executed at S1401. At S809, a first drawing process (H) is executed.

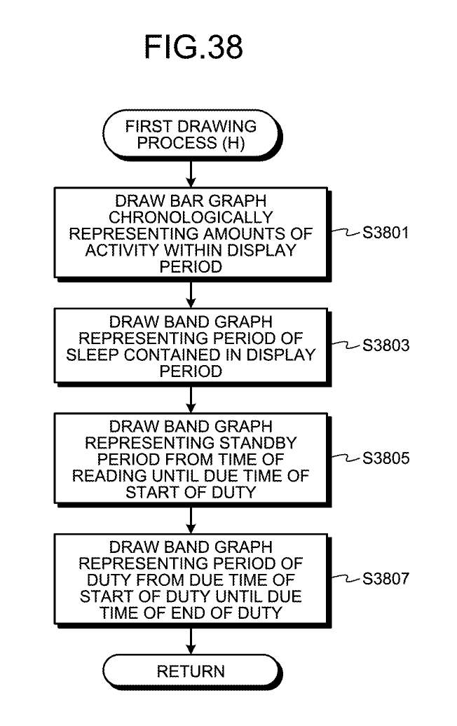

FIG. 38 illustrates a flow of the first drawing process (H). The drawing unit 409 draws the bar graph 301 chronologically representing amounts of activity within a display period (S3801). The drawing unit 409 draws the band graph 1301 representing the periods of sleep in the display period (S3803). The drawing unit 409 draws the band graph 2551 representing the standby period until a due time of the start of duty from a time of reading (S3805). The drawing unit 409 draws the band graph 3301 representing the period of duty from the due time of the start of duty until a due time of the end of duty (S3807). On ending the first drawing process (H), return to the main process (A) that is the caller process.

At S811, the display processing unit 411 displays the drawn graphs. In the tenth embodiment, the display processing unit 411 causes the display device 455 to display the bar graph 301, the band graph 1301, the band graph 2551 and the band graph 3301 that are drawn in the first drawing process (H).

The tenth embodiment is utilized to predict the body conditions of the user on standby and on duty based on the period in which the user is on standby and the period in which the user is on duty.

### [k] Eleventh Embodiment

In an eleventh embodiment, the first time band and the second time band are discriminated and the band graph 2551 of a standby period and the band graph 3301 of a period of duty are represented.

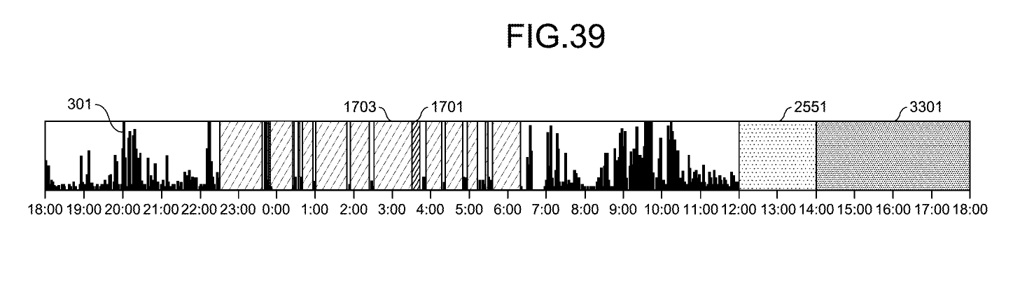

FIG. 39 represents an exemplary graph in the eleventh embodiment. In addition to the bar graph 301, the band graph 1701, the band graph 1703, the band graph 2551 and the band graph 3301 are represented.

In the eleventh embodiment, the main process (B) is executed. Furthermore, the first specifying process (C) is executed at S805 and the second specifying process (B) is executed at S1401. At S809, a first drawing process (I) is executed.

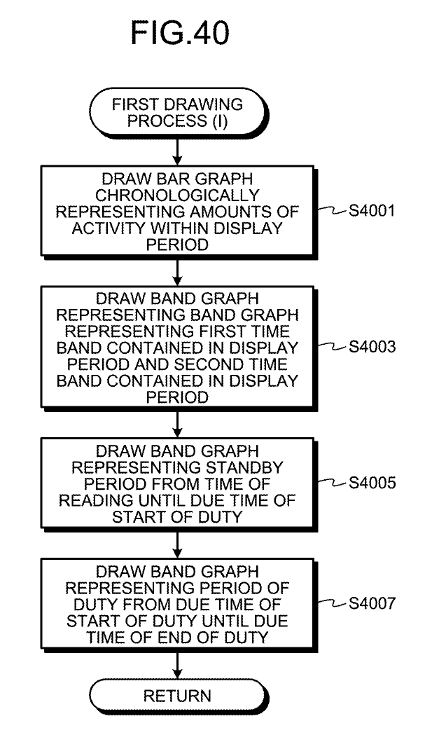

FIG. 40 represents a flow of the first drawing process (I). The drawing unit 409 draws the bar graph 301 chronologically representing amounts of activity within a display period (S4001). The drawing unit 409 draws the band graph 1701 representing the first time band contained in the display period and the band graph 1703 representing the second time band (S4003). The drawing unit 409 draws the band graph 2551 representing a standby period from a time of reading until a due time of the start of duty (S4005). The drawing unit 409 draws the band graph 3301 representing the period of duty from the due time of the start of duty until a due time of the end of duty (S4007). On ending the first drawing process (I), return to the main process (B) that is the caller process.

At S811, the display processing unit 411 displays the drawn graphs. In the eleventh embodiment, the display processing unit 411 causes the display device 455 to display the bar graph 301, the band graph 1701, the band graph 1703, the band graph 2551 and the band graph 3301 that are drawn in the first drawing process (I).

According to the eleventh embodiment, the depth of sleep can be taken into consideration to predict the body conditions of the user on standby and on duty.

### [l] Twelfth Embodiment

In a twelfth embodiment, body positions of the user are represented by band graphs.

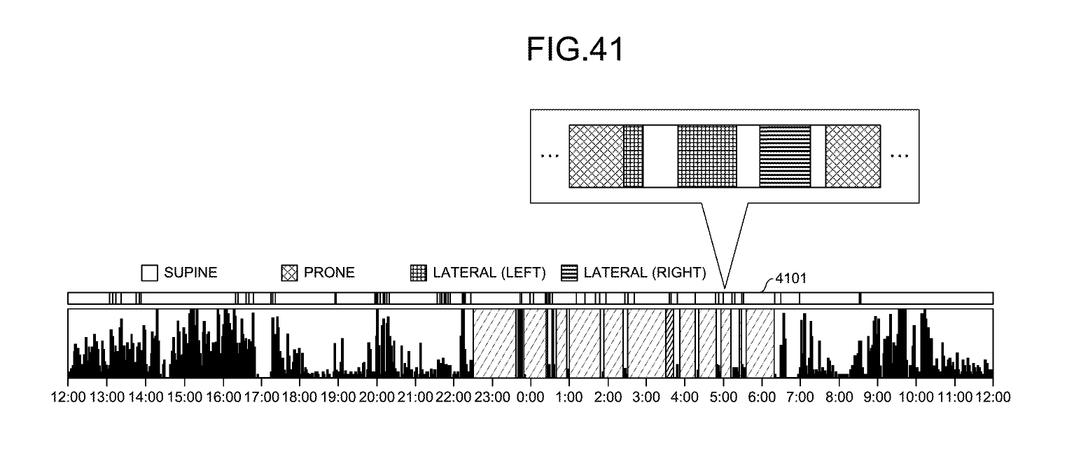

FIG. 41 represents an exemplary graph in the twelfth embodiment. A second graph area is provided above a first graph area. In the second graph area, a band graph 4101 representing body positions of the user is displayed.

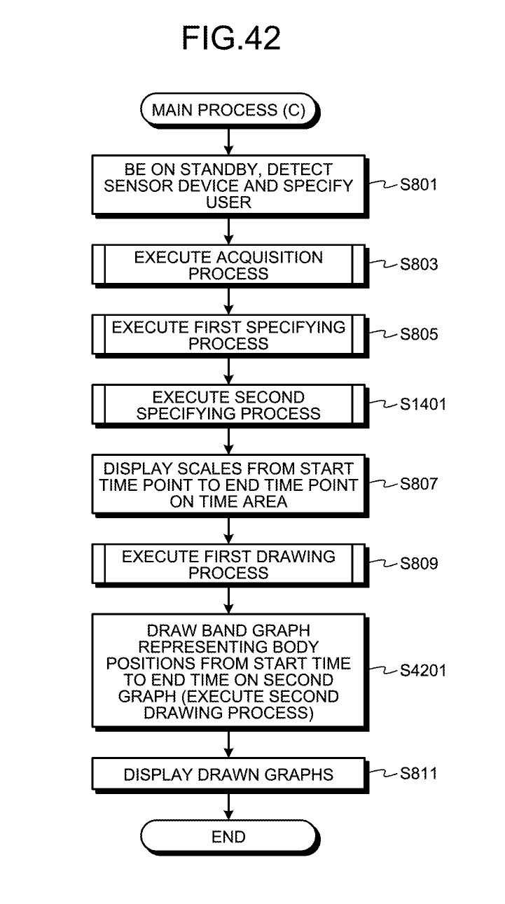

In the twelfth embodiment, a main process (C) is executed. FIG. 42 illustrates a flow of a main process (C). The drawing unit 409 executes the second drawing process (S4201). In the second drawing process, the band graph 4101 representing body positions in a display period is drawn in the second graph area. At S811, the display processing unit 411 displays the graphs that are drawn in the second drawing process together with the graphs that are drawn in the first drawing process. The graphs drawn in the first drawing process and the graphs that are drawn in the second drawing process match in a time axis.

The graph in FIG. 41 represents an exemplary case where the first specifying process (A) is executed at S805, the second specifying process (B) is executed at S1401 and the first drawing process (C) is executed at S809.

The main process (C) is based on the main process (B) and the second drawing process is added thereto. Alternatively, the main process (C) may be based on the main process (A) and the second drawing process may be added thereto. In other words, the twelfth embodiment may be applied to any of the above-described embodiments.

According to the twelfth embodiment, it is possible to estimate the condition of the user more in detail according to a combination of amounts of activity and body positions.

### [m] Thirteenth Embodiment

In a thirteenth embodiment, duration of a first period from a time point when the user awakes until a due time of the start of duty is displayed.

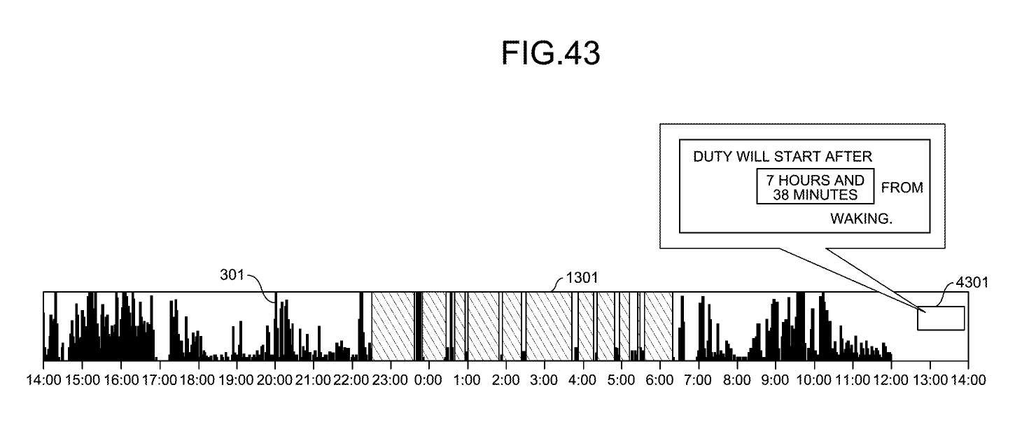

FIG. 43 represents an exemplary graph in the thirteenth embodiment. In the thirteenth embodiment, a window 4301 representing the first period is displayed together with graphs. In the example, the window 4301 is displayed near the due time of the start of duty. Note that the window 4301 may be displayed in another position. The first period may be displayed in a mode other than the window 4301.

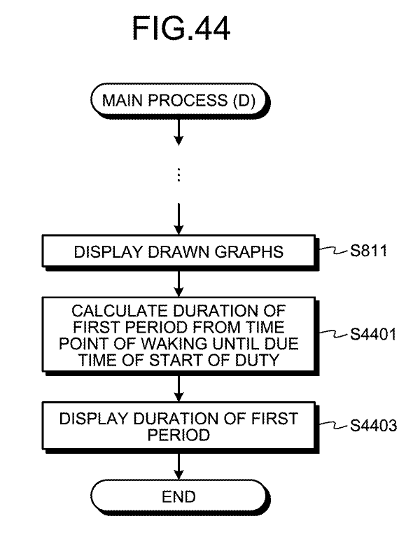

In the thirteenth embodiment, a main process (D) is executed. FIG. 44 illustrates a flow of the main process (D). The main process (D) may presuppose any one of the main processes (A) to (C). Following the process at S811, the first calculating unit 413 calculates the duration of the first period from the time point when the user awakes until the due time of the start of duty (S4401). The time point when the user awakes indicates the timing at which the condition is last switched from the sleep condition to the awake condition. The display processing unit 411 then displays the window 4301 representing the duration of the first period (S4403). The main process (D) then ends.

The graph in FIG. 43 represents an exemplary case where the first specifying process (B) is executed at S805, the second specifying process (A) is executed at S1401 and the first drawing process (B) is executed at S809. Note that the thirteenth embodiment may be applied to any one of the first to twelfth embodiments.

The thirteenth embodiment is useful to predict the body condition at the time point of the start of duty according to the time elapsing from the time point when the user awakes.

### [n] Fourteenth Embodiment

In a fourteenth embodiment, duration of a second period from a time point when the user awakes until a due time of the end of duty is displayed.

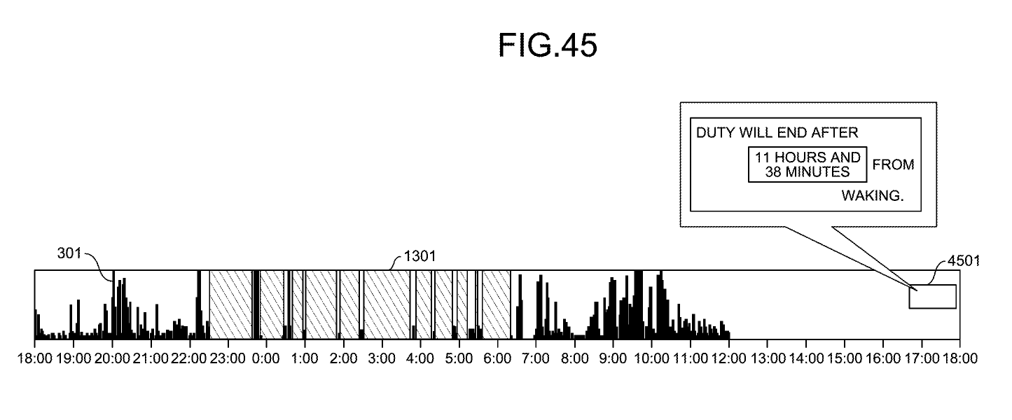

FIG. 45 represents an exemplary graph in the fourteenth embodiment. In the fourteenth embodiment, a window 4501 representing the second period is displayed together with the graph. In this example, the window 4501 is displayed near the due time of end of duty. Note that the window 4501 may be displayed in another position. The second period may be displayed in a mode other than the window 4501.

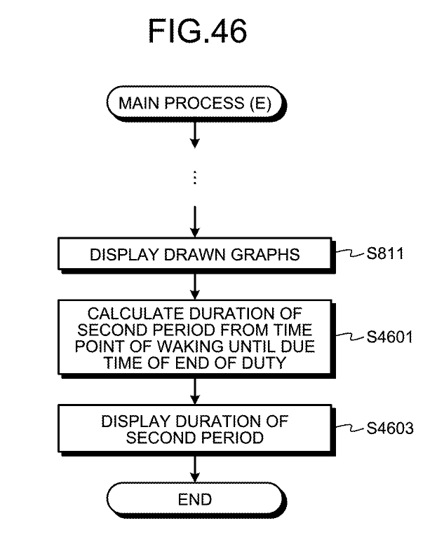

In the fourteenth embodiment, a main process (E) is executed. FIG. 46 illustrates a flow of the main process (E). The main process (E) presupposes any one of the main processes (A) to (C). Following the process at S811, the second calculating unit 415 calculates the duration of the second period from the time point when the user awakes until the due time of the end of duration (S4601). The display processing unit 411 displays the window 4501 representing the duration of the second period (S4603). The main process (E) then ends.

The graph in FIG. 45 represents an exemplary case where the first specifying process (C) is executed at S805, the second specifying process (A) is executed at S1401 and the first drawing process (B) is executed at S809. Note that the fourteenth embodiment may be applied to any of the first to twelfth embodiments.

The fourteenth embodiment is useful to predict the body condition at the time point of the end of duty according to the time elapsing from the time point when the user awakes.

### [o] Fifteenth Embodiment

In a fifteenth embodiment, duration of the first period and duration of the second period are displayed.

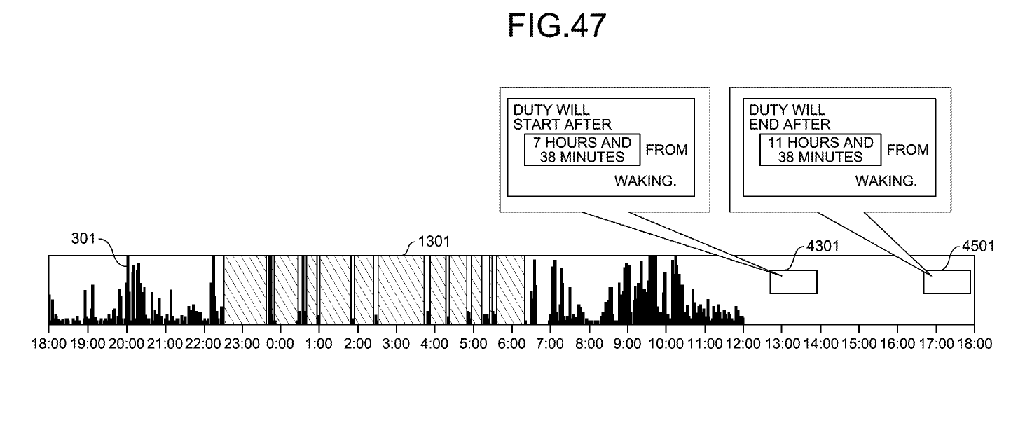

FIG. 47 represents an exemplary graph in the fifteenth embodiment. In the fifteenth embodiment, the window 4301 representing the first period and the window 4501 representing the second period are displayed together with graphs. The window 4301 and the window 4501 may be at any positions.

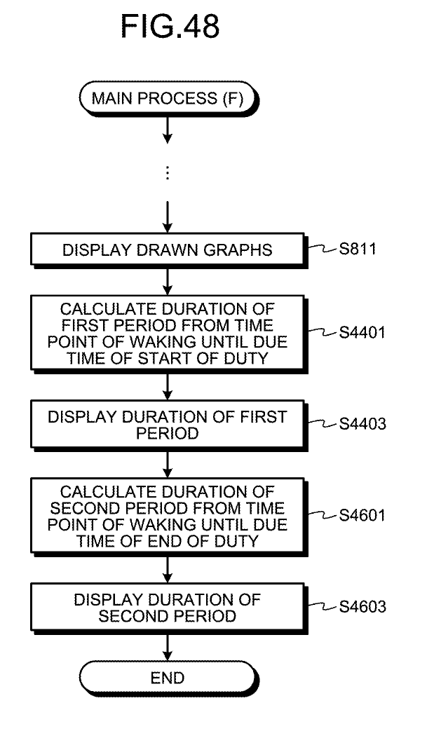

In the fifteenth embodiment, a main process (F) is executed. FIG. 48 illustrates a flow of the main process (F). The main process (F) may presuppose any one of the main processes (A) to (C). Following the process at S811, the first calculating unit 413 calculates the duration of the first period from the time point when the user awakes until the due time of the start of duty (S4401). The display processing unit 411 displays the window 4301 representing the duration of the first period (S4403).

The second calculating unit 415 further calculates the duration of the second period from the time point when the user awakes until a due time of the end of duty (S4601). The display processing unit 411 displays the window 4501 representing the duration of the second period (S4603).

The graph in FIG. 47 illustrates the exemplary case where the first specifying process (C) is executed at S805, the second specifying process (A) is executed at S1401, and the first drawing process (B) is executed at S809. The fifteenth embodiment may be applied to any of the first to twelfth embodiments.

The fifteenth embodiment is useful to predict the body conditions at the time point of the start of duty and the time point of the end of duty according to the time elapsing from the time point when the user awakes.

The diagram of each of the graphs represents an example where the areas are discriminated by the patterns with which the areas are filled; however, the areas may be discriminated by the colors with which the areas are filled.

The embodiments of the present invention have been described; however, the present invention is not limited thereto. For example, the above-described functional block configuration does not necessarily match the program module configuration.

The configurations of the respective storage areas described above are an example only and the configurations are not necessarily the above-described ones. In the process flows, the turns of processes may be switched or multiple processes may be executed in parallel as long as the processing results do not change.

The above-described information processing device 201 is a computer device. As illustrated in FIG. 49, the information processing device 201 includes a memory 2501, a central processing unit (CPU) 2503, a hard disk drive (HDD) 2505, a display controller 2507 that is connected to a display device 2509, a drive device 2513 for a removable disk 2511, an input device 2515 and a communication controller 2517 for connection to a network that are connected via a bus 2519. An operating system (OS) and an application program for performing the processes in the embodiments are stored in the HDD 2505 and, when executed by the CPU 2503, are read from the HDD 2505 into the memory 2501. The CPU 2503 controls the display controller 2507, the communication controller 2517 and the drive device 2513 to cause them to perform given operations according to the content of processes of the application program. The data being processed is stored mainly in the memory 2501. Alternatively, the data may be stored in the HDD 2505. In the embodiments of the present invention, the application programs for performing the above-described processes are stored in the computer-readable removable disk 2511 and distributed and then are installed from the drive device 2513 into the HDD 2505. The application program may be installed in the HDD 2505 via a network, such as the Internet, and the communication controller 2517. Such a computer device realizes the various functions like those described above in a way that hardware, such as the CPU 2503 and the memory 2501 described above, and programs, such as the OS and the application program, organically cooperate.

The above-described embodiments of the invention are summarized as follows.

A sensor display device according to the present embodiment includes (A) a first specifying unit configured to, when acquiring sensor values that are acquired and stored in a storage from a wearable sensor that stores the sensor values in the storage via a read device, automatically specify sensor values within a given time width stretching back from any one of first timing of reading, second timing that is due timing at which a user who is associated with the sensor starts duty, and third timing that is due timing at which the user ends duty and (B) a display device configured to chronologically display the specified sensor values or values that are calculated based on the sensor values.

Accordingly, it is possible to provide a display device capable of displaying a screen from which the condition of the user before given timing is estimated easily.

The sensor values or the calculated values may be time series data about states of activity of the user who wears the wearable sensor. The sensor display device may include a second specifying unit configured to specify a sleep period during which the user sleeps based on the time series data; a drawing unit configured to draw a first graph representing the states of activity and the sleep period within the given time width; and a display processing unit configured to cause the display device to display the first graph.

Accordingly, it is possible to provide a display device capable of displaying a screen from which the body condition of the user is estimated easily.

The display processing unit may further cause the display device to display the first graph whose end is the second timing.

Accordingly, it is possible to provide the display device capable of displaying a screen from which a period stretching back from the start of duty is known easily. From such a screen, for example, a period from a time point when the user awakes until the start of duty is known easily.

The drawing unit may be further configured to draw an image representing a period from the first timing until the second timing on the first graph.

Accordingly, it is possible to provide a display device capable of displaying a screen from which an effect of the standby on the body condition is predicted easily based on a period during which the user is on standby.

The display processing unit may be further configured to cause the display device to display the first graph whose end is the third timing.

Accordingly, it is possible to provide a display device capable of displaying a screen from which a period stretching back from the end of duty is known easily. From such a screen, for example, the period from the time point when the user awakes until the end of duty is known easily.

The drawing unit may be further configured to draw an image representing a period from the second timing until the third timing on the first graph.

Accordingly, it is possible to provide a display device capable of displaying a screen from which the body condition of the user on duty is predicted easily based on the period during which the user is on duty.

The second specifying unit may be further configured to specify a first time band in which the user has a light sleep and a second time band in which the user has a deep sleep in the sleep period. The drawing unit may be configured to draw the first time band and the second time band on the first graph in different modes.

Accordingly, it is possible to provide a display device capable of displaying a screen from which the state of sleep of the user is known easily.

The drawing unit may be further configured to draw a second graph chronologically representing body positions of the user and the display processing unit may be further configured to cause the display device to display the second graph according to a time axis of the first graph.

Accordingly, it is possible to provide a display device capable of displaying a screen from which the condition of the user is estimated more in detail easily according to combinations of the states of activity and body positions.

The sensor device may further include a first calculating unit configured to calculate duration of a first period from the time point when the user awakes until the second timing. The display processing unit may be further configured to cause the display device to display the duration of the first period.

Accordingly, it is possible to provide a display device capable of displaying a screen from which the body condition at the time point of the start of duty is predicted easily according to the time elapsing from the time point when the user awakes.

The sensor display device may further include a second calculating unit configured to calculate duration of the second period from the time point when the user awakes until the third timing. The display processing unit may be configured to cause the display device to display the duration of the second period.

Accordingly, it is possible to provide a display device capable of displaying a screen from which the body condition of the user on duty is predicated easily according to the time elapsing from the time point when the user awakes.

Furthermore, the given time width may be 24 hours.

This enables easy analysis on the body condition based on a day cycle. It is estimated that the period of sleep is contained in the 24 hours as long as a complete overnight stay is not performed.

A program for causing the computer to perform the processes perfoLmed by the above-described sensor display device can be created, and the program may be stored in a computer-readable storage medium or a storage device, such as a flexible disk, a CD-ROM, a magneto-optical disk, a semiconductor memory or a hard disk. Note that, generally, intermediate results of processes are stored temporarily in a storage device, such as a main memory.

According to an aspect, it is possible to make an output from which the condition of a user before given timing is estimated easily.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. An information processing device comprising:
a memory; and
a processor coupled to the memory and configured to:
specify part of sensor values from among the sensor values which is acquired from a wearable sensor when acquiring the sensor values stored in a storage of the wearable sensor via a read device, the part of sensor values corresponding to a certain time period stretching back from any one of first timing of reading from the wearable sensor, second timing that is due timing at which a user who is associated with the wearable sensor starts duty, and third timing that is due timing at which the user ends duty; and
chronologically control a display device to display the part of sensor values or calculated values that are calculated based on the part of sensor values.

2. The information processing device according to claim 1, wherein
the sensor values or the calculated values are time series data relating to states of activity of the user who wears the wearable sensor, and
the processor is further configured to:
specify a sleep period during which the user sleeps based on the time series data;
draw a first graph representing the states of activity and the sleep period within the certain time period; and
control the display device to display the first graph.

3. The information processing device according to claim 2, wherein the processor is further configured to control the display device to display the first graph whose end is the second timing.

4. The information processing device according to claim 2, wherein the processor is further configured to draw an image representing a time period from the first timing until the second timing on the first graph.

5. The information processing device according to claim 2, wherein the processor is further configured to control the display device to display the first graph whose end is the third timing.

6. The information processing device according to claim 2, wherein the processor is further configured to draw an image representing a time period from the second timing until the third timing.

7. The information processing device according to claim 2, wherein the processor is further configured to:
specify a first time band in which the user has a sleep and a second time band in which the user has another sleep in the sleep period, the another sleep being deeper than the sleep; and
draw the first time band and the second time band on the first graph in different modes.

8. The information processing device according to claim 2, wherein the processor is further configured to:
draw a second graph chronologically representing body positions of the user; and
control the display device to display the second graph associating with a time axis of the first graph.

9. The information processing device according to claim 2, wherein the processor is further configured to:
calculate duration of a first period from a time point when the user awakes until the second timing; and
control the display device to display the duration of the first period.

10. The information processing device according to claim 2, wherein the processor is further configured to:
calculate duration of a second period from a time point when the user awakes until the third timing; and
control the display device to display the duration of the second period.

11. The information processing device according to claim 1, wherein the certain time period is set for 24 hours.

12. A sensor display method executed by a processor, the sensor display method comprising:
specifying part of sensor values from among the sensor values which is acquired from a wearable sensor when acquiring the sensor values stored in a storage of the wearable sensor via a read device, the part of sensor values corresponding to a certain time period stretching back from any one of first timing of reading from the wearable sensor, second timing that is due timing at which a user who is associated with the wearable sensor starts duty, and third timing that is due timing at which the user ends duty; and
chronologically controlling a display device to display the part of sensor values or calculated values that are calculated based on the part of sensor values.

13. A non-transitory computer-readable recording medium storing therein a sensor display program that causes a computer to execute a process, the process comprising:
specifying part of sensor values from among the sensor values which is acquired from a wearable sensor when acquiring the sensor values stored in a storage of the wearable sensor via a read device, the part of sensor values corresponding to a certain time period stretching back from any one of first timing of reading from the wearable sensor, second timing that is due timing at which a user who is associated with the wearable sensor starts duty, and third timing that is due timing at which the user ends duty; and
chronologically controlling a display device to display the part of sensor values or calculated values that are calculated based on the part of sensor values.
